(19) United States
(12) Patent Application Publication
(10) Pub. No.: US 2006/0088844 A1
(43) Pub. Date: Apr. 27, 2006

(54) Real-time PCR microarray based on evanescent wave biosensor

(75) Inventor: Liang Xu, Shanghai Spring City (CN)

Correspondence Address: HONEYWELL INTERNATIONAL INC., 101 COLUMBIA ROAD, P O BOX 2245, MORRISTOWN, NJ, 07962-2245, US

(73) Assignee: Honeywell International Inc., Morristown, NJ (US)

(21) Appl. No.: 10/972,033

(22) Filed: Oct. 22, 2004

(51) Int. Cl. C12Q 1/68; C12M 1/34

(52) U.S. Cl. 435/6; 435/287.2

(57) ABSTRACT

A system and method for simultaneous, quantitative measurement of nucleic acids in a sample. Fluorescently tagged amplicons of the target nucleic acids are localized on a substrate surface by hybridization to oligopobes that have been arrayed and tethered to the substrate surface in a pre-determined, two-dimensional pattern. The hybridized, amplicons are then detected by exciting their fluorescent tags using an evanescent wave of light of the appropriate wave-length. Because of the limited penetration of the evanescent wave (about 100-300 nm), the fluorescently tagged nucleotides in the remainder of the reaction cell do not fluoresce. By measuring the fluorescence at various locations on the substrate surface, the current abundance of hybridized amplicons of each of the target nucleic acids can be determined. The analytic techniques of real time PCR may then be used to obtain accurate, quantitative measurements for each of the nucleic acids in the sample.

### FIELD OF THE INVENTION

The present invention relates to systems and methods for quantitative measurement of nucleic acids, and particularly to systems and methods for the real-time, simultaneous quantitative assay of a plurality of nucleic acids.

### BACKGROUND OF THE INVENTION

The quantitative assay of nucleic acids is of considerable importance in basic biological research as well as in fields such as clinical microbiology. A quantitative assay is typically accomplished in two stages. The target nucleic acid in a sample is first amplified to produce a detectable amount of nucleic acid for use by quantifying tools. The detected amount of a target nucleic acid is then used to calculate the amount of that nucleic acid that was initially present in the sample.

The polymerese chain reaction (PCR) is a powerful way of amplifying nucleic acids, particularly deoxyribonucleic acid (DNA). The key to practical PCR is the use of a thermostable DNA polymerase, i.e., a protein capable of catalyzing DNA replication that does not denature at the elevated temperatures required to separate a DNA helix into two single strands of nucleic acid.

PCR is initiated by placing a target double stranded DNA in a buffer of nucleotides along with a supply of small sequences of single stranded DNA, known as primers, which are complementary to the target DNA and a thermostable DNA polymerase. By cycling the temperature of the mixture through three stages, the target DNA can be exponentially amplified. The first stage is a high temperature (94 degrees Centigrade) denaturing stage, in which double stranded DNA is separated into two single strands. The second stage is a low temperature (60 degrees Centigrade) annealing stage, in which the primers bind to the single stranded DNA. The final, extension stage occurs at an intermediate temperature (72-78 degrees Centigrade). In the extension stage, the DNA polymerase catalyzes the extension of primers that have annealed to single strands of target DNA, adding appropriate nucleotides until a complete, double stranded DNA helix is formed. In each PCR cycle, the number of copies of the target DNA approximately doubles, allowing for rapid accumulation of the target DNA.

In principle, the quantity of a target DNA produced at the end of a series of PCR cycles (also known as the “end product”) is proportional to the number of copies of that target DNA in the initial sample. However, in practice, the exponential nature of the amplification, and subtleties of the primer annealing that initiates the replication, result in saturation and other effects that make the PCR end product a very unreliable estimate of the amount of a target DNA in the initial sample.

The real time polymerase chain reaction (real time PCR) process was developed in the mid 1990's to improve the original PCR process in a way that avoids these difficulties and provides reliable, accurate quantitative measurements of the number of copies of any target DNA in the sample. In a real time PCR, fluorogenic probes that are only active when bound to target DNA are added to the PCR buffer solution. These fluorogenic probes are single strands of DNA, with a middle portion having a sequence of nucleotides that is complementary to the target DNA. On either side of this middle portion, are extension nucleotide sequences that are complementary to each other, so that an unattached probe will fold onto itself in a hairpin configuration. The fluorogenic probe has a fluorescent molecule at one end, and a fluorescence quenching molecule at the other end. An unattached, folded probe therefore has a fluorescing and a quenching molecule adjacent to each other, and consequently no fluorescent light is emitted when the unattached probe is illuminated. When the fluorogenic probe is attached to its target DNA, however, it is unfolded, with the fluorescing and quenching molecules separated from each other. When the attached probe is illuminated with the appropriate wavelength of light, the fluorescent molecule therefore emits fluorescent light.

By providing sufficient fluorogenic probes for a particular target DNA, and measuring the fluorescence from the bound probes at each stage of the PCR reaction, the number of amplicons at each stage of the reaction can be measured. This measurement can then be used to very accurately determine the number of copies of the DNA in the initial sample because of a straight line relationship between the fractional number of cycles for the number of amplicons to reach a pre-determined threshold and the logarithm of the number of copies in the initial sample.

In this way, real time PCR may be used to determine the amount of a target DNA in a sample with less than 2% error over a range of 9 orders of magnitude, i.e., it can count as few as 5, and as many as 5 billion, strands of the target DNA copies in the initial sample.

Real time PCR technology does, however, have limitations, the most significant of which is that real time PCR can only measure a small number of nucleic acid in one reaction tube to date since a limited number of suitable fluorescent dyes with suitable corresponding, fluorescence exciting light sources.

For many applications, the simultaneous quantification of more than one kind of nucleic acid is highly desirable. What is needed is an apparatus and method that allows real time PCR to be used to simultaneously quantify hundreds of different nucleic acids using a small number of fluorescent dyes, and preferably only one fluorescent dye.

### SUMMARY OF THE INVENTION

The present invention provides a system and method for simultaneous, quantitative measurement of a plurality of nucleic acids in a sample.

In an exemplary embodiment, the nucleic acids in the sample are all amplified in a single reaction cell using a polymerase chain reaction (PCR), reverse transcription PCR, roll cycle replication, or T7 transcription linear amplification, in which the amplification buffer solution additionally contains fluorescently-tagged nucleotides or fluorescently-tagged primer, so that the amplicons of the target nucleic acids are themselves fluorescently tagged.

During the annealing and/or extension phases of the amplification process, the fluorescently tagged amplicons of the target nucleic acids are localized onto a substrate surface by hybridization with oligopobes that have been arrayed and tethered to the substrate surface in a pre-determined, two-dimensional pattern. The oligoprobes have the complementary, nucleotide sequence as the target nucleic acids and may be arrayed by robotic printing using commercially available microarraying technology.

The hybridized, fluorescently tagged target amplicons are then detected by the fluorescence emitted when their fluorescent tags are exited by an evanescent wave of light of the appropriate wave-length. Because the evanescent wave decays exponentially as it enters the reaction cell, with an effective range of about 100-300 nm, it only penetrates far enough into the reaction cell to activate fluorescent tags very close to the substrate surface, i.e., the fluorescently tagged target amplicons hybridized to the oligopobes tethered to the surface. The evanescent wave does not, therefore, activate the fluorescently tagged nucleotides in the remainder of the reaction cell.

By monitoring the strength of the fluorescence at the various locations on the substrate surface, the current abundance of hybridized amplicons of each of the target nucleic acids can be determined. This may be done in real time as the PCR reaction progresses, and the analytic techniques of real time PCR then used to obtain accurate, quantitative measurements of the abundance of each of the target nucleic acids in the original sample.

These and other features of the invention will be more fully understood by references to the following drawings.

### BRIEF DESCRIPTION OF THE DRAWINGS

FIG. 1 is a cross-sectional view of an exemplary cartridge capable of evanescent wave detection of fluorescently tagged amplicons in a microarrayed PCR reaction in an initial stage of the PCR process.

FIG. 2 is cross-sectional view of an exemplary cartridge capable of evanescent wave detection of fluorescently tagged amplicons in a microarrayed PCR reaction at the end of the annealing and extension stage of the PCR process.

FIG. 3 is cross-sectional view of an exemplary cartridge capable of evanescent wave detection of fluorescently tagged amplicons in a microarrayed PCR reaction at the denaturation stage of the PCR process.

FIG. 4 is cross-sectional view of an exemplary cartridge showing evanescent wave detection of fluorescently tagged amplicons in a microarrayed PCR reaction at the detection stage of the PCR process.

FIG. 5 are exemplary plots of fluorescent intensity against PCR cycle.

FIG. 6 is an exemplary plot of the logarithm of the number of copies of target DNA strands, log[N], in the original sample against the threshold cycle, CT, for that target DNA.

### DETAILED DESCRIPTION

The present invention provides a system and method capable of real-time, simultaneous, quantitative measurement of a plurality of nucleic acids in a sample.

In an exemplary embodiment, the nucleic acids in the sample are amplified using the polymerase chain reaction (PCR). The PCR is a well known method of amplifying one or more stands of deoxyribonucleic acid (DNA), begun by placing the target DNA in a buffer containing primer DNA, the nucleotides adenine (A), thymine (T), cytosine (C) and guanine (G) (collectively referred to as dNTPs), a DNA polymerase and primers. The primers are short strands of DNA, with sequences that complement one end of a nucleic acid to be amplified. The primers initiate replication of that target DNA.

The PCR process has three main steps: denaturation, annealing and extension. In the denaturation step, the mixture is heated to about 94 degrees Celsius, at which temperature all the DNA separates into single strands. The mixture is then quickly cooled. As the temperature falls to about 60 degrees Centigrade , the annealing step occurs, in which the primers hybridize or bind to their complementary sequences on the target DNA. The temperature is then raised to be within the optimal 72-78 degrees Centigrade range for the extension step. In this step, the DNA polymerase uses the dNTPs in solution to extend the annealed primer, and form a new strand of DNA known as an amplicon. The amplicon is a complementary copy of the original target DNA strand, and is initially bound to it in a double helix configuration. The mixture is then briefly reheated back to about 94 degrees Centigrade to separate the newly created double helix stands into single strands of nucleic acid, and so begin another cycle of the PRC process. With each cycle of the PCR process, the number of copies of the original target DNA roughly doubles.

In a preferred embodiment of the present invention, the PCR buffer additionally contains fluorescently-tagged dNTPs, i.e., dNTPs having a fluorescent dye molecule attached to them, so that upon completion of each PCR cycle, the amplicons produced are fluorescently tagged. The amplicons of the target DNA are then localized, using probe strands of DNA known as oligoprobes. The oligoprobes have the complementary, nucleotide sequence as the target DNA. The oligopobes are tethered to a substrate surface in a known, two-dimensional pattern, with the substrate surface forming part of the reaction cell containing the PCR ingredients.

During the annealing and extension phases of the PCR process, the fluorescently-tagged, target amplicons hybridize to their corresponding oligoprobes. The hybridized, fluorescently tagged target amplicons are then illuminated with an evanescent wave of light of the appropriate wave-length to activate the fluorescent dye molecules of the tagged dNTPs. This evanescent wave decays exponentially in power after entering the reaction cell via the substrate surface to which the oligoprobes are tethered, with an effective penetration range of about 300 nm. This means that the evanescent wave penetrates far enough into the reaction cell to activate the fluorescently tagged amplicons hybridized to those oligopobes, but that it does not activate the fluorescently tagged dNTPS in solution in the main body of the reaction cell. By monitoring the strength of the fluorescence at various locations on the substrate surface, the current abundance of amplicons of the corresponding, target DNA can be determined. This may be done in real time as the PCR reaction progresses, and the results used to obtain a quantitative measure of the abundance of a specific target in the original sample, in a manner analogous to the real time PCR calculation.

An exemplary embodiment of the method will now be described in more detail by reference to the accompanying drawings in which, as far as possible, like numbers refer to like elements.

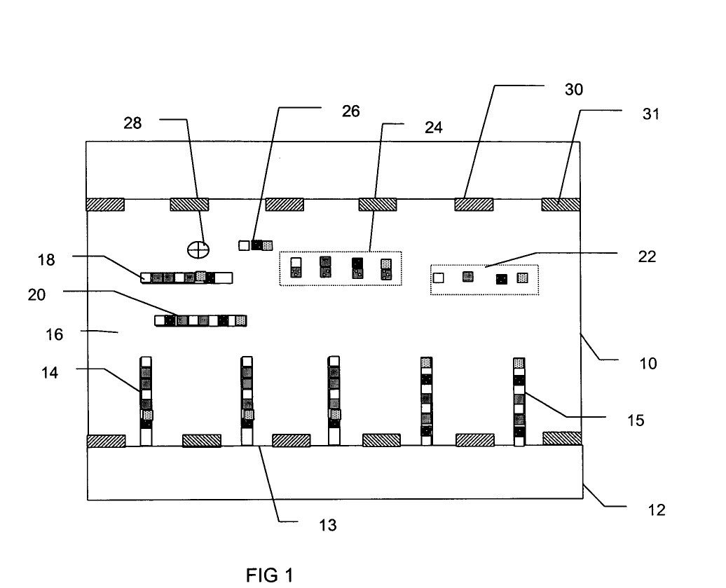

FIG. 1 is a cross-sectional view of an exemplary reaction cartridge capable of evanescent wave detection of fluorescently tagged amplicons in a microarrayed PCR reaction, comprising a reaction cartridge 10, a substrate 12 having a surface 13, a first oligoprobe 14, a second oligoprobe 15, a buffer solution 16, a first DNA strand 18, a second DNA strand 20, dNTPs 22, fluorescently tagged dNTPs 24, a primer 26, a thermostable DNA polymerase 28, a heating element 30 and a cooling element 31.

In a preferred embodiment, the substrate 12 is comprised of a material that is optically denser than the buffer solution 16, so that evanesant wave detection can be used as described in detail below. The substrate 12 may for instance be glass, or a suitably coated plastic or polymer.

First and second oligoprobes 14 and 15 are strands of DNA, each having a specific nucleotide sequence of one of the target strands of DNA 18 and 20 that they are used to detect. In a preferred embodiment these oligoprobes are non-extendable. In other words, the nucleotides cannot be added to either end of the oligoprobes. Oligoprobes 14 and 15 may be natural or synthetically fabricated polynucleotides, polynucleotides with artificial bases and/or artificial carbohydrates, peptide nucleic acids (“PNA”s), bicyclic nucleic acid, or other nucleotide analogs, sconstructed using a commercially available oligonucleotide synthesizer such as, but not limited to, the Polyplex® synthesizer available from Genomic Solutions, Inc. of Ann Arbor, Mich., or they may be, but not limited to, a sequence choosen from a library of DNA sequences, such as a library of expressed sequence tags (EST) known to have some biological significance.

The oligoprobes 14 and 15 are arrayed on a substrate surface 13. In a preferred embodiment, oligoprobes 14 and 15 are arrayed on the substrate as small spots by robotic printing using commercially available microarraying technology such as, but not limited to, the Omnigrid® microarrayer available from Genomic Solutions, Inc. of Ann Arbor, Mich.

The oligoprobes may be immobilized on the substrate surface by one of the well-known techniques such as, but not limited to, covalently conjugating an active silyl moiety onto the oligoprobes. Such silanized molecules are immobilized instantly onto glass surfaces after manual or automated deposition. Alternately the oligoprobes may be immobilized by suitably electrically charging the surface, preferably by using a suitable coating such as, but not limited to, silane or poly-L-lysine.

Fluorescently tagged dNTPs 24 are nucleotides tagged with a fluorescent dye such as, but not limited to, fluorescein or Rhodamine Green dyes, or similar, related compounds having similar fluorescing characteristics, such as functionalized or intercalating dyes and luminescent, functionalized nanoparticles (“quantum dots”). dNTPs 24 may have one, two, three or four of the four base nucleotides dGTP, dCTP, dATP and dTTP fluoresently tagged. In a preferred embodiment, only one of the nucleotides is tagged, e.g. only dCTP.

Heating elements 30 may be any suitable resistive material such as, but not limited to, carbon, that provides heat when an electric current flows though it. Heating elements 30 need to be capable of heat the reaction cell to 94 degrees Centigrade within minutes. Cooling elements 31 may be any suitable solid state cooling element such as, but not limited to, a well known Peltier solid-state device functioning as a heat pump. The heating elements and cooling elements can also be outside the cartridge.

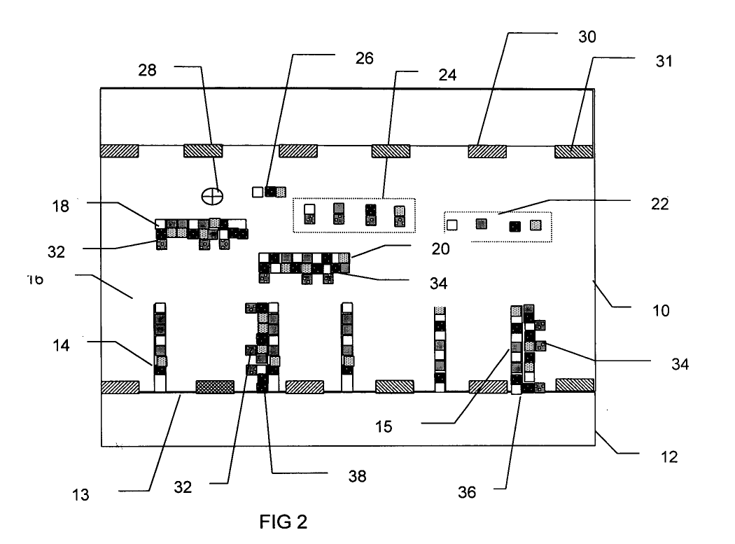

FIG. 2 is cross-sectional view of an exemplary cartridge capable of evanescent wave detection of fluorescently tagged amplicons in a microarrayed PCR reaction at the end of the annealing and/or extension stage of the PCR process, further comprising first and second fluorescently tagged amplicon 32 and 34. First fluorescently tagged amplicon 32 is a DNA strand having a nucleotide sequence that is complementary copy of the first target DNA strand 18, i.e., for every adenine (A) nucleotide in the first target DNA strand 18, there is a thymine (T) nucleotide in the first amplicon 32, and vice versa. Similarly for every cytosine (C) nucleotide in the first target strand 18, there is a guanine (G) nucleotide in the first amplicon 32.

At the end of the annealing and/or extension stage of the PCR process, the amplicons 32 and 34 produced by extension of annealed primers 26 remain hybridized to their corresponding target DNA strands 18 and 20. Additionally, amplicons produced in previous cycles of the PCR process are hybridized to the tethered oligoprobes 14 and 15. For instance, at surface site 36, a second fluorescently tagged amplicon 34 is hybridized to a second oligoprobe 15. Similarly at surface site 38, a first fluorescently tagged amplicon 32 is hybridized to a first oligoprobe 15. The oligoprobes 14 and 15 are designed not to be amplified in the PCR process by, for instance, being tethered by their 3′ end to the substrate, or by having the 3′ end modified by dideoxidation or by having a stable group added to the 3′ end or by any other well known methods of making oligoprobes not participate in a PCR process in the presence of specific primers.

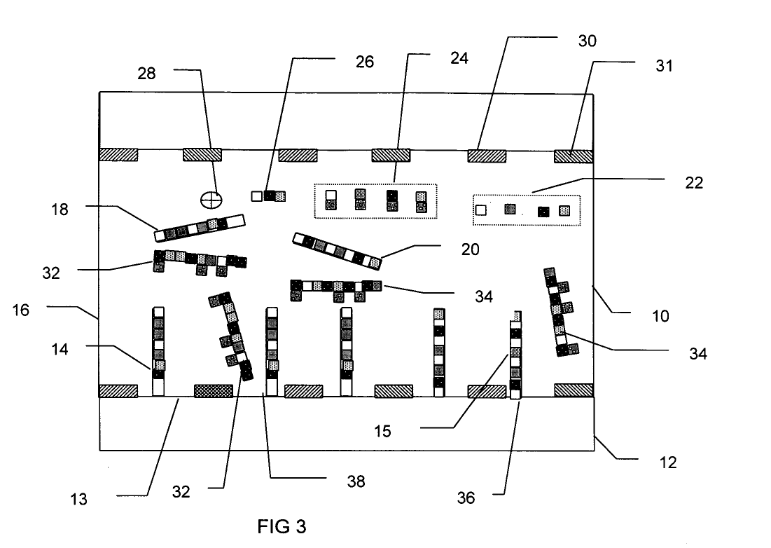

FIG. 3 is cross-sectional view of an exemplary cartridge capable of evanescent wave detection of fluorescently tagged amplicons in a microarrayed PCR reaction at the denaturation stage of the PCR process. In this stage, the mixture within the reaction cell 12 has been heated to close to 100 degrees Centigrade, and optimally to about 94 degrees Centigrade. At this temperature, the DNA is denatured, i.e., it separates into individual, single strands. When cooled in the next stage of the PCR process, each of the individual DNA target strands 18 and 20, as well as each of the fluorescent amplicons 32 and 34, will anneal with first primers 26. The annealed primers 26 will then be extended as the thermostable DNA polymerase 28 adds appropriate nucleotides, until each individual DNA target strand 18 and 20, and each fluorescent amplicons 32 and 34, will be hybridized to a new amplicon which is a copy or a complementary copy of the original target strands 18 and 20.

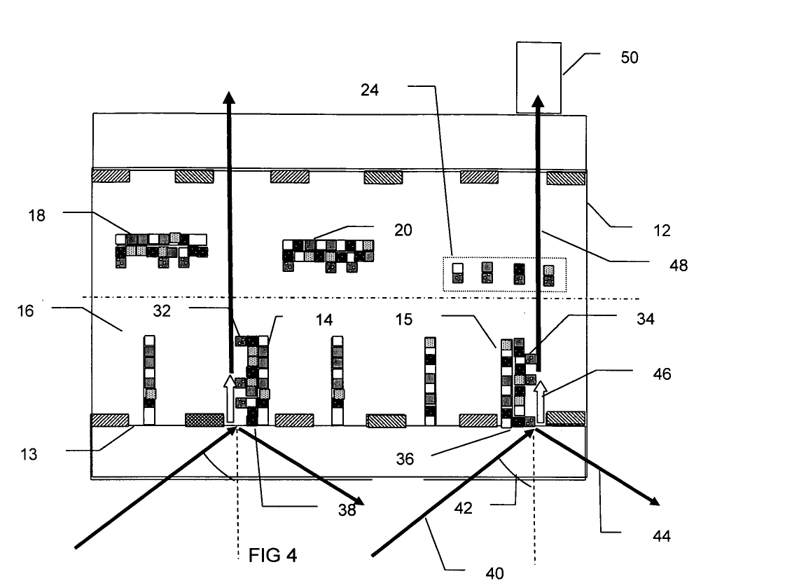

FIG. 4 is cross-sectional view of an exemplary cartridge showing evanescent wave detection of fluorescently tagged amplicons in a microarrayed PCR reaction at the detection stage of the PCR process, further comprising an incident beam of light 40, an angle of incidence 42, a reflected beam of light 44, an evanescent beam of light 46, a fluorescent beam of light 48 and a fluorescent light detector 50. The detection stage can be coincident with the annealing and/or extension stage.

The incident beam of light 40 is chosen to be of a wavelength suitable for exiting the flurophore used to label the dNTPs 24. In a preferred embodiment, the incident beam of light 40 is the 488 nm spectral line of an argon-ion laser, which closely matches the excitation maximum (494 nm) of fluorescein dye that is preferably used to tag dNTPs 24.

The angle of incident 42 of the incident beam of light 40 is chosen to be greater than the critical angle of the substrate to buffer interface. The critical angle of incidence is the angle at which total internal reflection occurs and is dependent on the refractive indices of the materials forming the interface. From Snell's laws of refraction,
Critical angle of incidence=sin⁻¹(n₁/n₂)
where n₁ and n₂ are the refractive indices of the materials on either side of the interface. In a preferred embodiment of the present invention, the substrate 12 is comprised of glass and has a refractive index of about 1.5, while the buffer 16 is comprised mainly of water having a refractive index of about 1.3, so that the critical angle of incidence is about 61 degrees.

When light is reflected off an interface 13 at an angle of incidence 42 greater than the critical angle so that total internal reflection occurs, an evanescent wave 46 is formed and propagates through the interface. The intensity of the evanescent wave 46 drops by a factor of e for each 130 nm increase in distance from the interface. Thus only objects very near the interface are illuminated by the evanescent wave 46. This property is used in a preferred embodiment of the present invention to preferentially illuminate the first and second fluorescently tagged amplicons 32 and 34 that are hybridized to the first and second oligoprobes 14 and 15. The fluorescent light 48 emitted by the fluorescently tagged amplicons 14 and 15 may then be detected and analyzed by the fluorescent light detector 50. The fluorescent light detector 50 typically comprises collection optics such as, but not limited to, a microscope objective lens, which focuses the light on to a detection system such as, but not limited to, a photomuliplier tube or a charge coupled device (CCD) camera or photodiodes.

The origin and intensity of the collected fluorescent light can then be used to estimate the number of fluorescently emitting molecules and therefore the number of fluorescently tagged amplicons currently hybridized to a particular type of oligoprobe using, for example, the well known quantification techniques employed in Real Time or Kinetic PCR analysis. In these, the reactions are characterized by the point in time during cycling when amplification of a PCR product is first detected, rather that the amount of PCR product accumulated after a fixed number of cycles. The higher the number of copies of a nucleic acid target in the initial sample, the sooner a significant increase in fluorescence is observed.

In a further embodiment of the invention, the fluorescent signal may be detected by monitoring the reflected light and determining the amount of light absorbed by the fluorescent tags.

FIG. 5 are exemplary plots 52, 54 and 56 of fluorescent signal verses the cycle number for three target DNA strands, each having a different number of copies in the initial sample. There is a starting or baseline, background fluorescence signal, detectable even when no PCR cycle has taken place. In the initial cycles of the PCR, there is little change in this fluorescence signal. An increase in fluorescence above the baseline indicates the detection of accumulated PCR product. By setting a fixed fluorescence threshold above the baseline, a threshold cycle (CT) parameter can be defined as the fractional cycle number at which the fluorescence for a particular oligoprobe passes this fixed threshold, as indicated by the three fractional values Cᵣ₁, Cᵣ₂ and Cᵣ₃.

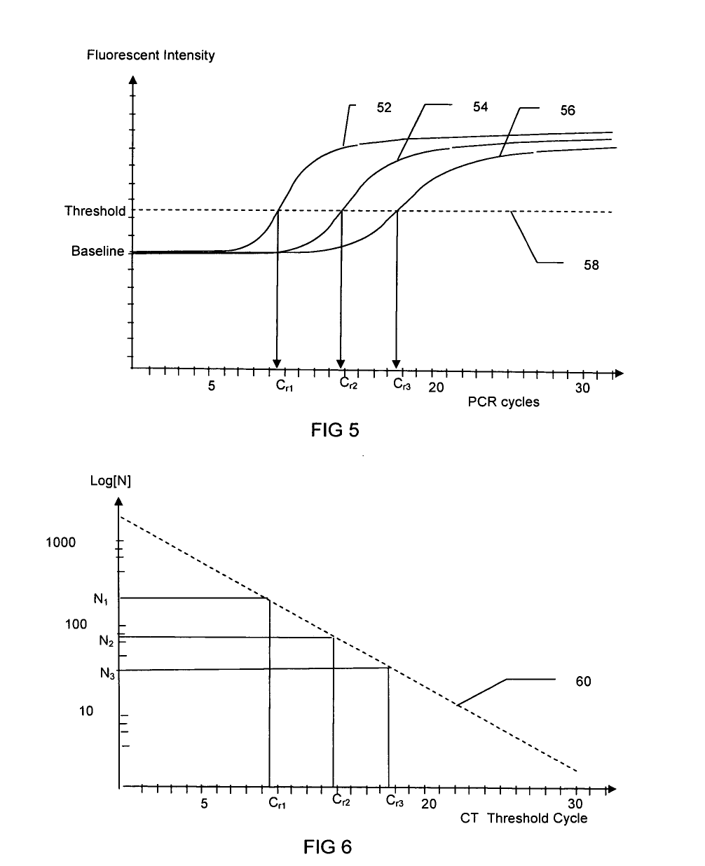

FIG. 6 is an exemplary plot of the logarithm of the number of copies of target DNA strands, log[N], in the original sample against the threshold cycle, CT, for that target DNA. Because of the exponential nature of the PCR, a plot of the log of the initial target copy number verses CT is a straight line 60. By introducing a number of calibration DNA targets, having a known number of copies in the initial sample, the fluorescence associated with their corresponding oligoprobes can be used to produce a straight line calibration line 60 of log of initial copy number verses CT. By measuring the CT of a location on the reaction cell known to have a particular oligoprobe, the number of copies of the target DNA corresponding to that oligoprobe can then be deduced from the calibration curve.

Although the foregoing discussion has used DNA as an exemplary nucleic acid, it would be obvious to a person of reasonable skill in the art to apply the invention to other nucleic acids, including RNA sequences or combinations of RNA and DNA sequences.

Although the foregoing discussion has used PCR as an exemplary reaction, it would be obvious to one of ordinary skill in the art to apply the methods of the invention using any suitable amplification reaction such as, but not limited to, reverse transcription PCR, random primer amplification, roll cycle amplification or linear amplification “T7”.

Although the foregoing discussion has used fluorescent tagged dNTP to label the target DNA, it would be obvious to one of ordinary skill in the art to use related structures such as, but not limited to, fluorescent tagged primers, functionalized nanoparticles, or intercalating dyes to label the target DNA.

Although the foregoing discussion has, for simplicity, been discussed in terms of two target nucleic acids, it would be obvious to one of ordinary skill in the art to use the methods and apparatus for the quantitative evaluation of one target nucleic acid, or for a multiplicity of target nucleic acids.

Although the invention has been described in language specific to structural features and/or methodological acts, it is to be understood that the invention defined in the appended claims is not necessarily limited to the specific features or acts described. Rather, the specific features and acts are disclosed as exemplary forms of implementing the claimed invention.

What is claimed is:

1. A method of quantitatively analyzing a target nucleic acid, comprising the steps of:
providing a fluorescently tagged amplicon of said target nucleic acid;
providing a substrate having an upper surface;
providing an oligoprobe in close proximity to said upper surface of said substrate;
annealing said fluorescently tagged amplicon to said oligoprobe;
activating a fluorescence from said fluorescently tagged amplicon hybridized to said oligopobe, using an evanescent wave of a predetermined wavelength;
detecting said fluorescence for quantitative analysis of said target nucleic acid.

2. The method of claim 1, wherein providing a fluorescently tagged amplicon comprises the steps of providing a fluorescently tagged nucleotide and performing a cycle of an amplification reaction comprising the steps of denaturing, annealing and extending.

3. The method of claim 2, wherein annealing said fluorescently tagged amplicon to said oligoprobe occurs during said annealing step of said polymerase chain reaction.

4. The method of claim 2, wherein said step of detecting said fluorescence occurs during said annealing or extending step of said amplification reaction.

5. The method of claim 1, wherein said step of providing an oligoprobe in close proximity to said substrate further includes the step of printing said oligoprobe onto said substrate using a micro-array printer; and immobilizing said oligoprobe onto said substrate.

6. The method recited in claim 5, wherein said step of immobilizing said oligoprobe further includes positively charging said substrate.

7. The method recited in claim 6, wherein said step of positively charging further includes coating said substrate with a reagent chosen from the group comprising silane, avidin, or poly-L-lysine, or a combination thereof.

8. The method of claim 1 wherein said amplification reaction is a real time polymerase chain reaction.

9. An apparatus for quantitatively analyzing a target nucleic acid, comprising:
a substrate having an upper and a lower surface and a refractive index greater than a refractive index of water;
a buffer solution substantially in contact with said upper surface of said substrate, said buffer solution being capable of sustaining an amplification reaction and containing a fluorescently tagged nucleotide and said target nucleic acid;
an oligoprobe close proximity to said upper surface of said substrate and within said buffer solution, said oligoprobe having a nucleotide sequence corresponding to, or complementary to, a nucleotide sequence of said target nucleic acid;
a ray of light, having a wavelength chosen to activate said fluorescent tag, incident on an interface between said substrate and said buffer solution at an angle chosen so that an evanescent wave propagates into said buffer solution;
a detector capable of detecting fluorescent light emitted by said fluorescent tag.

10. The apparatus of claim 9, further comprising a heating element capable of cycling a temperature of said buffer solution, thereby enabling said amplification reaction.

11. The apparatus of claim 9 wherein said amplification reaction is a real time polymerase chain reaction.
